(19) United States
(12) Patent Application Publication
(10) Pub. No.: US 2012/0157763 A1
(43) Pub. Date: Jun. 21, 2012

(54) METHOD AND APPARATUS FOR REDUCING TISSUE TRAUMA DURING IPP PROCEDURE

(76) Inventor: Ryan Darnell, Eden Prairie, MN (US)

(21) Appl. No.: 13/328,701

(22) Filed: Dec. 16, 2011

Related U.S. Application Data
(60) Provisional application No. 61/423,658, filed on Dec. 16, 2010.

(51) Int. Cl. A61F 5/41

(52) U.S. Cl. 600/40

(57) ABSTRACT

A traction ring for use in a penile implant procedure can include a plurality of elastic sutures. Each elastic suture can have an elastic body with an attachment loop at one end and an adhesive pad at the opposed end. The attachment loop allows for positioning of the elastic suture at any of a plurality of available attachment grooves located about a perimeter of the traction ring. The adhesive pad is generally attached to penis proximate the penis tip. Adhesive pad can be bonded to the penis with a suitable skin adhesive. The skin adhesive can be contained within a reservoir on the adhesive pad. Alternatively, the adhesive pad can be separated from the elastic body. A removal solvent can be applied to dissolve or otherwise break down the skin adhesive following completion of an implant procedure.

### RELATED APPLICATION

The present application claims the benefit of U.S. Provisional Application No. 61/423,658 filed Dec. 16, 2010, and entitled, “PENILE PROSTHESIS SURGICAL METHODS AND APPARATUS”, which is incorporated herein in its entirety by reference.

### FIELD OF THE INVENTION

The present invention relates generally to a surgical method and apparatus for minimizing trauma to the penis during a prosthetic implantation procedure. More specifically, the present invention is directed to a traction device utilizing an adhesive pad so as to avoid tissue punctures and reduce potential infection points.

### BACKGROUND OF THE INVENTION

Advertisements for pharmaceutical treatments for impotence have become ubiquitous. More and more men and their partners now are seeking treatment for impotence. In the recent past, it was estimated that only one in twenty sufferers of erectile dysfunction sought treatment from their doctors. Pharmaceutical treatments are successful for only a subset of impotence sufferers. Other treatments, including more invasive treatments are necessary for many men. These treatments include injection therapy, vacuum devices and penile prosthesis.

Penile implants are disclosed in U.S. Pat. Nos. 3,853,122; 4,009,711; 3,954,102; 4,537,183; 4,566,466; 3,987,789, 4,350,151; 5,788,627; 4,651,721; 5,141,509; 5,851,176; 5,167,611; 6,443,887 and 6,533,719. Commercial examples of penile prosthesis include the AMS 700 CX™ and the AMS 700 Ultrex™ available from American Medical Systems of Minnetonka, Minn.

The penile implant has been used for decades and provides a selected and reliable erection. Inflatable penile implants typically include a pair of cylinders. In some instances, these cylinders are connected to a fluid-filled reservoir with a pump and valve assembly. The two cylinders are normally implanted into the corpus cavernosae of the patient's penis and the reservoir is typically implanted into the patient's abdomen. The pump assembly is implanted in the scrotum. During use, the patient actuates the pump and fluid is transferred from the reservoir through the pump and into the cylinders. This results in the inflation of the cylinders and produces rigidity for a normal erection. Then, when the patient desires to deflate the cylinders, a valve assembly within the pump is actuated in a manner such that the fluid in the cylinders is released back into the reservoir. This deflation returns the penis to a flaccid state.

Prior art methods for implanting inflatable prostheses typically included the step of suspending the patient's penis from a traction ring using a hook as illustrated in FIG. 1. The hook engages the head of the penis by penetrating the tissue. As a result, there are two puncture wounds in the penis that require care and healing following the procedure. There is a desire to reduce the trauma and associated risks of this implantation surgery.

### SUMMARY OF THE INVENTION

The invention addresses the trauma and associated risks of penile prosthesis implantation. In one embodiment, the penis is suspended from a traction ring by an adhesive pad. The pad is removed after the surgical procedure. Thus no punctures of the skin at end of penis are created, there is a reduced exposure to infection points and the patient has fewer wounds to heal.

In one aspect of the present invention, an embodiment of a traction ring can include a plurality of elastic sutures. Each elastic suture have an elastic body with an attachment loop at one end and an adhesive pad at the opposed end. The attachment loop allows a medical professional to orient the elastic suture any one of a plurality of available attachment grooves located about a perimeter of the traction ring. Adhesive pad is generally attached to penis proximate the penis tip to provide the appropriate tension. Adhesive pad can be bonded to the penis with a suitable skin adhesive. In some embodiments, the skin adhesive can be contained within a reservoir on the adhesive pad. Alternatively, the adhesive pad can be separated from the elastic body such that the adhesive can be applied to the penis, the elastic body can be placed into contact with the adhesive and the adhesive pad can placed onto the adhesive whereby the elastic body is captured. In some embodiments, a removal solvent can be applied to dissolve or otherwise break down the adhesive such that the adhesive pad can be removed from the penis following completion of an implant procedure.

In another aspect of the present invention, an implant kit can be provided that includes a traction ring of the present invention along with a penile prosthetic device.

The above summary of the various representative embodiments of the invention is not intended to describe each illustrated embodiment or every implementation of the invention. Rather, the embodiments are chosen and described so that others skilled in the art can appreciate and understand the principles and practices of the invention. The figures in the detailed description that follow more particularly exemplify these embodiments.

### BRIEF DESCRIPTION OF THE DRAWINGS

The invention can be completely understood in consideration of the following detailed description of various embodiments of the invention in connection with the accompanying drawings, in which:

FIG. 1 is a schematic illustration of a penis suspended from a traction ring according to the prior art.

FIG. 2 is a plan view of a penile implant of the prior art.

FIG. 3 is a schematic illustration of a penis suspended from a traction ring according to an embodiment of the present invention.

FIG. 4 is a partial schematic illustration of a penis suspended from the traction ring of FIG. 3.

FIG. 5 is a section view of an adhesive pad according to an embodiment of the present invention.

FIG. 6 is plan, partial hidden view of an adhesive pad according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF PREFERRED EMBODIMENTS

Various types and sizes of implantable penile prostheses are available for treatment of erectile dysfunction, and various specialized tools exist for implanting such prostheses. A typical penile prosthesis includes at least one pair of cylinders that are inflatable and are each implantable in one of the corpus cavernosa. The penile prosthesis further includes a pump external to the cylinder for pressurizing the cylinder. The pump is typically connected to the cylinder through a tubing near the proximal end of the cylinder. Each cylinder has a fluid tube connected thereto between the ends at a position spaced further from the distal end and closer to the proximal end thereof. A pump is disposed in the scrotum, and connected through tubing to a spherical abdominal reservoir. The reservoir is placed in the pelvic area near the urinary bladder behind the muscles of the abdominal wall. The cylinders are inflated as fluid is pumped from the reservoir, and are deflated as fluid is transferred back to the reservoir. This inflation and deflation allows the patient to control whether his penis is erect or flaccid. An example of such penile prosthesis is the AMS 700™ inflatable penile prosthesis manufactured by American Medical Systems, Inc. Further illustrative of the devices available is that disclosed in U.S. Pat. No. 4,590,927, which is hereby incorporated by reference, that relates to a unitary penile prosthesis which comprises a tubular enclosure having a distal portion which includes a pump, a medial portion including a pressurizable chamber which contains an internal tubular, substantially non-distensible portion and a concentric tubular sleeve, and a proximal portion defining a fluid reservoir therewithin.

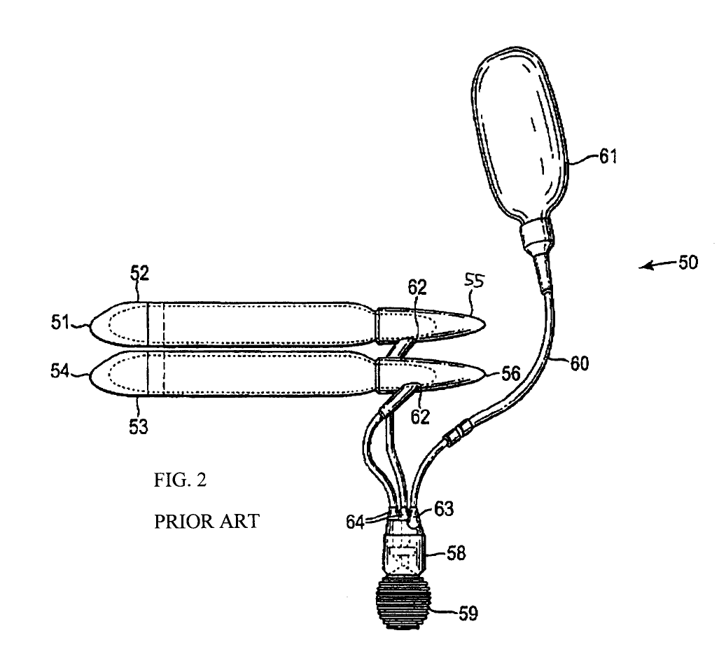

As shown in FIG. 2, a penile implant according to the present invention such as that disclosed in U.S. Pat. No. 3,954,102 to Buuck and U.S. Pat. No. 4,424,807 to Evans, Sr., and U.S. Pat. No. 5,263,981 to Polyak et al., all of which are herein incorporated by reference, basically comprises a prosthetic device 50 including a pair of cylinders 51 and 54, a valve and pump assembly 58, a fluid reservoir 61, and intercommunicating flexible conduits 60. The entire device 50 is arranged to be surgically implanted in the body, with the two cylinders 51 and 54 being disposed within the penis, with the combination valve and pump assembly 58, disposed in either the abdominal cavity or in the scrotum and with the reservoir 61 disposed with the abdominal cavity.

Referring again to FIG. 2, the cylinders 51 and 54 are arranged for surgical implantation in respective corpus cavernosum and are constructed to mechanically expand the corpora cavernosa until the fibrous tissue envelope becomes tense, thereby producing a functional erection without the necessity for the corpora cavernosa to be engorged with blood. The cylinders are readily deflatable to enable the penis to become flaccid when an erection is no longer sought.

The cylinders 51 and 54 are placed within the respective corpora cavernosum by surgically preparing a passageway therein. The passageway can be formed by any conventional surgical technique used for prior art penile implants. The passageways can be of any suitable shape. Each passageway extends down a substantial portion of the length of the associated corporus cavernosum from a point adjacent the glans penis to a point adjacent the scrotum.

Each of the cylinders is an enclosed hollow member formed of a very thin, e.g., 2 5 mils, flexible membrane. As synthetic material, any appropriate material tolerated by the tissues may be used, and in particular silicone rubber. As can be seen clearly in FIG. 2, each cylinder is of generally cylindrical shape whose opposed ends are rounded or domed. The distal end of the cylinder is denoted by the reference numeral 52 and 55 and the proximal end by reference numerals 53 and 56. The distal end is arranged to be located adjacent the glans penis when the cylinder is located within the passageway and with the proximal end located adjacent the root of the penile shaft close to the scrotum. Each cylinder includes an opening or access port 62 to the interior thereof and which is located adjacent the proximal end of the cylinder.

The cylinders are arranged to be filled with a fluid, such as water, through its respective access port 62 to cause the cylinder to expand longitudinally, as well as radially. Since each cylinder is located in a respective passageway in a corpora cavernosa, the expansion of the cylinder causes concomitant expansion of the corpora cavernosa from its minimal volume (its “flaccid volume”) to an increased volume.

Each cylinder has sufficient volumetric capacity so that when it is inflated to the condition where the fibrous envelope surrounding the corpora cavernosa has reached the limit of its expansion, the material forming the membrane wall of the cylinder has not reached the point at which it undergoes tension, e.g., it does not begin to stretch.

The means for filling the cylinders 51 and 54 comprises the reservoir 61, the valve pump assembly 58 and the interconnecting tubing or conduits 60. To that end, the reservoir 61 is connected, via a tubing section, to one port 63 of the valve-pump assembly 58. The valve-pump assembly 58 comprises a squeezable bulb 59 coupled to interiorly located valves. The valve-pump assembly 58 also includes additional port(s) to which a conduit section is connected.

Operation of the device to produce an erection is as follows: The bulb 59 is squeezed through the skin of the scrotum, whereupon water from the reservoir 61 is forced through the port 63 and the tubing 64 to the associated access ports 62 of each of the cylinders. Thus, the cylinders begin to fill beyond the partially filled state. The pump bulb 59 has to be squeezed several times to effect a full erection. In this regard, each time that the pump bulb is squeezed, more water is forced into the cylinders through the conduit. Release of the pump or bulb 59 does not allow the water to return to the reservoir since the valve in the valve-pump assembly 58 is of the one-way type. Thus, each time the bulk is resqueezed, additional water is forced into the cylinders. This action causes the cylinders to expand from a flaccid condition to the fully filled or erect condition. In order to effect the filling of the cylinders, all that is required is pressure on the order of 2½ psi. As long as the valve precludes the water in the cylinders from flowing back to the reservoir, the cylinders remain in their fully filled state and the erection is maintained. When it is desired to render the penis flaccid, all that is required is to actuate a release mechanism (not shown) forming a portion of the valve-pump assembly 58 by squeezing on the scrotum where the release mechanism is located, whereupon the valve in the assembly 58 enables the water to flow out of the cylinder and into the reservoir under the natural pressure caused by the resiliency of the fibrous envelope on the cylinders. When the pressure produced on the water in the cylinders by fibrous tissue envelope equals the pressure on the water in the reservoir, the flow of water ceases. Thus, the pump bulb 59 must be squeezed to force the water back into the reservoir so that the cylinders are back in their partially deflated condition, whereupon the penis is limp and flaccid. Other prostheses and implantation methods are disclosed in U.S. Pat. Nos. 7,169,103, 6,929,599 and 7,066,878, all of which are fully incorporated herein.

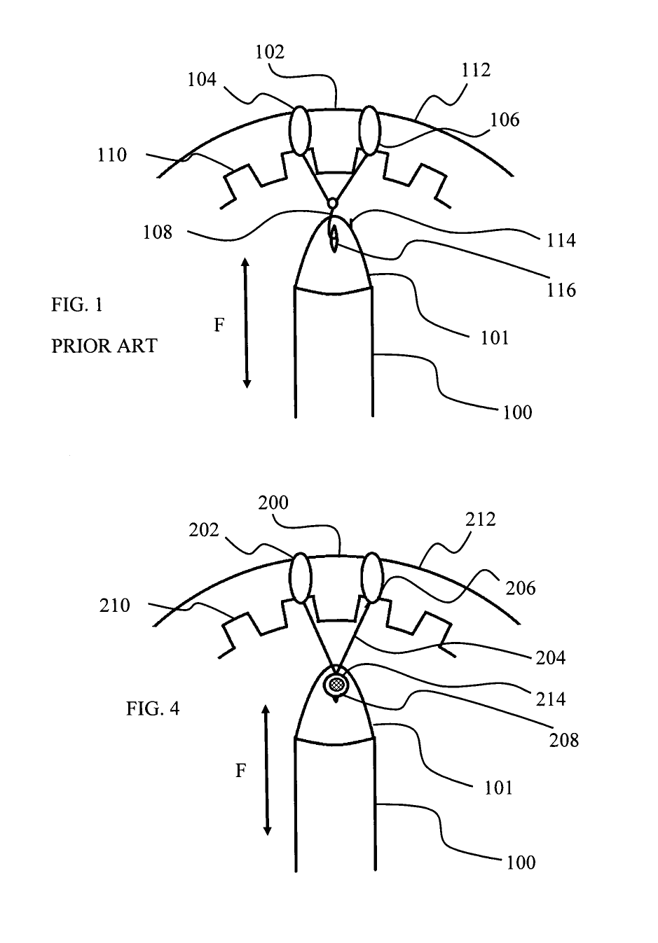

In preparing to implant the cylinders 51 and 54 within a penis 100, a surgeon typically captures, extends and retains the penis 100 utilizing a traction ring 102 positioned proximate the penis 100 as illustrated in FIG. 1. Traction ring 102 generally includes a plurality of elastic sutures 104, wherein each elastic suture generally includes an elastic body 105, an attachment loop 106 and a retention hook 108. A representative traction ring 102 includes the Lone Star line of reusable retractor rings available from Cooper Surgical of Trumbull, Conn. Attachment loop 106 allows the medical professional to adjust the location of the elastic suture 104 so as to provide the appropriate tension (indicated as arrow F) during a surgical procedure. Attachment loop 106 can be mounted in any one of a plurality of available attachment grooves 110 located about a perimeter 112 of the traction ring 102. Retention hook 108 is generally formed of a medically suitable material, such as stainless steel, and includes a hook tip 114 configured to puncture and exit through the penis 100 proximate a penis tip 101. Attachment of the retention hook 108 creates two puncture wounds 116 at the penis tip 101, which can be painful and unsightly during a recovery period as well as providing two entry points for bacteria that can lead to infection.

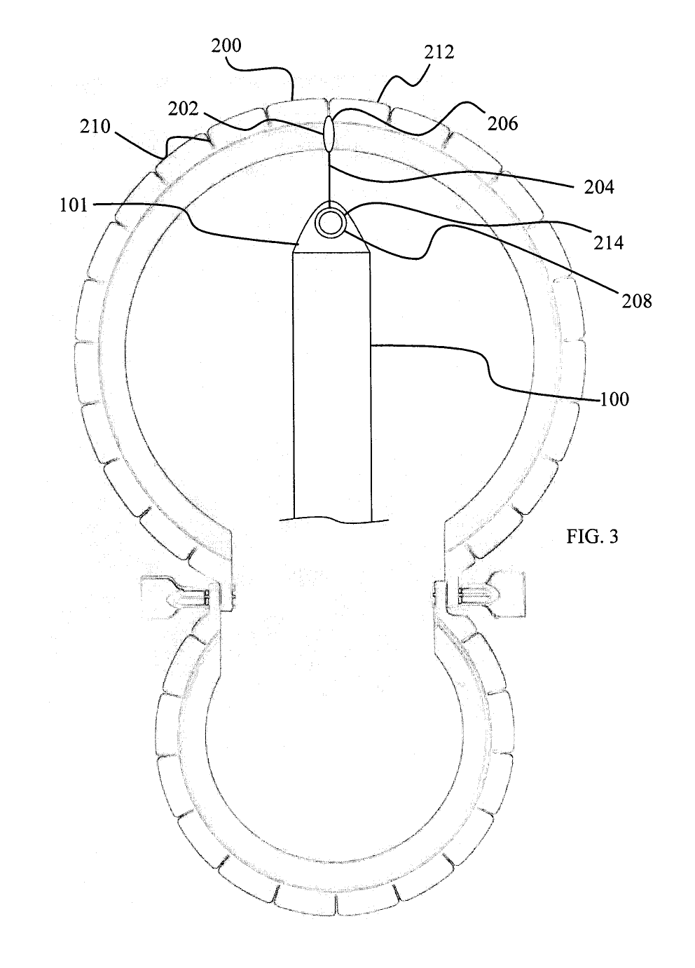

Referring to FIGS. 3 and 4, an embodiment of a traction ring 200 according to the present invention can include a plurality of elastic sutures 202. Each elastic suture 202 comprises an elastic body 204, an attachment loop 206 and an adhesive pad 208. Similar to attachment loop 106, attachment loop 206 allows a medical professional to orient the elastic suture 202 to any one of a plurality of available attachment grooves 210 located about a perimeter 212 of the traction ring 200. Adhesive pad 208 is generally attached to penis 100 proximate the penis tip 101 to provide the appropriate tension (indicated as arrow F).

Adhesive pad 208 is generally bonded to penis 100 using a suitable skin adhesive 214. Generally, suitable skin adhesives 214 will be fast acting, high strength and are generally non-toxic and suited for skin contact. Representative skin adhesives can include various polymers dissolved in a solvent, for example, water or alcohol. Representative skin adhesives can further comprise additional medicants such as, for example, antiseptics or the like. Suitable polymers can include, for example, polyvinylpyrrolidone (water based), pyroxylin/nitrocellulose or poly(methylacrylate-isobutene-monoisopropylmaleate) (alcohol based), and acrylate or siloxane polymers (hexamethyldisiloxane or isooctane solvent based). One especially suitable adhesive can be based on ethyle and methyl cyanoacrylates, which are found in liquid bandage products such as the brands Dermabond, Band-Aid and VetGlu.

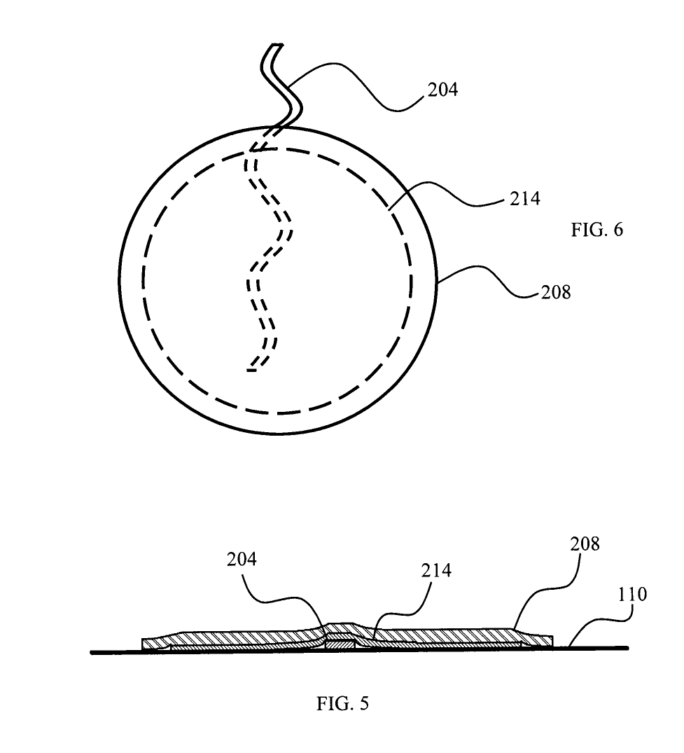

In some embodiments, adhesive pad 208 can incorporate a reservoir of skin adhesive 214 as shown in FIG. 5 that can be broken or otherwise punctured immediately prior to attaching the adhesive pad 208 to penis 100. In some embodiments, skin adhesive 214 can be directly applied to the penis 100 wherein the adhesive pad 208 is then pressed or otherwise placed into position on the skin adhesive 214. Finally, in certain embodiments, adhesive pad 208 can be physically separate from the elastic suture 202 as shown in FIG. 6 such that following placement of the skin adhesive 214 on the penis, the elastic body 204 is stretched and layed over the skin adhesive 214, whereby the adhesive pad is then placed on top of the elastic body 204 to sandwich and capture the elastic body 204 such that tension is applied to penis 100.

Following implantation of cylinders 51 and 54, a medical professional can carefully apply a removal solvent 216 on and around the adhesive pad 208 so as to dissolve or otherwise break/destroy the bonding properties of the skin adhesive 214. Representative removal solvents 216 can include acetone and/or acetone based products. Following application of the solvent removal 216, the elastic suture 202 releases retention of penis 100. Removal solvent 216 should generally be non-toxic and be suitable for topical skin applications.

Through the use of traction ring 200 including the adhesive pad 208, potential appearance and medical complication issues can be avoided. By avoiding the puncture wounds associated with retention hook 108, potential entry points for bacteria and infection can be avoided. In addition, physical discomfort and appearance issues associated with healing. In certain embodiments, prosthetic device 50 and traction ring 200 can be provided together in kit form such that the medical professional is provided everything necessary for successful implant procedure.

All patents, patent applications, and publications cited herein are hereby incorporated by reference in their entirety as if individually incorporated, and include those references incorporated within the identified patents, patent applications and publications.

Although specific examples have been illustrated and described herein, it will be appreciated by those of ordinary skill in the art that any arrangement calculated to achieve the same purpose could be substituted for the specific examples shown. This application is intended to cover adaptations or variations of the present subject matter. Therefore, it is intended that the invention be defined by the attached claims and their legal equivalents, as well as the following illustrative embodiments.

1. An IPP traction system, comprising:
a traction ring including a plurality of spaced apart sutures, wherein a first end of each suture is operably attached to the traction ring and a second end of each suture is operably attached to an adhesive pad, wherein the adhesive pad includes a contact surface including a high strength skin adhesive for externally bonding to a penis tip.

2. The IPP traction system of claim 1, wherein each of the spaced apart sutures comprises a flexible suture.

3. The IPP traction system of claim 1, further comprising:
a removal solvent for dissolving the high strength skin adhesive following completion of an IPP procedure.

4. The IPP traction system of claim 3, wherein the removal solvent comprises: acetone and acetone-based solvents.

5. The IPP traction system of claim 1, wherein the adhesive pad further comprises a reservoir of the high strength skin adhesive.

6. The IPP traction system of claim 1, wherein the high strength skin adhesive comprises a polymer selected from polyvinylpyrrolidones, pyroxylin/nitrocellulose, poly(methylacrylate-isobutene-monoisopropylmaleate), acrylate or siloxane polymers and ethyl and methyl cyanoacrylates.

7. A method for reducing infection during an IPP procedure, comprising:
providing a traction ring including a plurality of spaced apart sutures, wherein a
first end of each suture is operably attached to the traction ring and a second end of each suture is operably attached to an adhesive pad;
placing the adhesive pad of each suture proximate a penis tip, such that a contact surface of the adhesive pad is in contact with the penis tip, wherein a high strength skin adhesive operably attaches the adhesive pad to the penis tip; and
retaining the penis tip with the adhesive pad of each suture such that a penis is maintained in a stretched condition during implantation of an IPP device.

8. The method of claim 7, further comprising:
applying a removal solvent to the penis tip whereby each adhesive pad can be detached from the penis tip.

9. The method of claim 7, further comprising:
puncturing a reservoir of high strength skin adhesive located on the adhesive pad prior to placing the adhesive pad proximate the penis tip.

10. A kit for performing an IPP procedure, comprising:
an IPP device; and
a traction ring including a plurality of spaced apart sutures, wherein a first end of each suture is operably attached to the traction ring and a second end of each suture is operably attached to an adhesive pad, wherein the adhesive pad includes a contact surface including a high strength skin adhesive for bonding to a patient's skin surface.

11. The kit of claim 10, wherein the IPP device, further comprises:
a prosthetic device, a pair of cylinders, a valve and pump assembly, a fluid reservoir and intercommunicating flexible conduits.

12. The kit of claim 11, wherein each of the spaced apart sutures comprises a flexible suture.

13. The kit of claim 11, further comprising:
a removal solvent for dissolving the high strength skin adhesive following completion of an IPP procedure.

14. The kit of claim 13, wherein the removal solvent comprises: acetone and acetone-based solvents.

15. The kit of claim 11, wherein the adhesive pad further comprises further comprises a reservoir of the high strength skin adhesive.

16. The kit of claim 11, wherein the high tensile adhesive comprises a polymer selected from polyvinylpyrrolidones, pyroxylin/nitrocellulose, poly(methylacrylate-isobutene-monoisopropylmaleate), acrylate or siloxane polymers and ethyl and methyl cyanoacrylates.
